(19) United States
(12) Patent Application Publication
(10) Pub. No.: US 2026/0263704 A1
(43) Pub. Date: Sep. 10, 2026

(54) STERILE SAFETY HYPODERMIC NEEDLE WITH SAFETY SHIELD

(71) Applicant: Azur Medical Company, Inc., Richmond, VA (US)

(72) Inventor: Leqi Zhao, Arcadia, CA (US)

(21) Appl. No.: 19/561,090

(22) Filed: Mar. 9, 2026

Related U.S. Application Data
(60) Provisional application No. 63/769,493, filed on Mar. 10, 2025.

(51) Int. Cl. A61M 5/32

(52) U.S. Cl. CPC A61M 5/3202

(57) ABSTRACT

A needle protection device can include a collar, a needle cover, and a cap. The collar can be securable to a hub or a barrel of a syringe, and the collar can include one or more projections. The needle cover can be releasably securable to the collar and can be configured to at least partially surround a needle of the syringe when the needle cover is secured to the collar. The cap can be pivotably connected to the collar and can be movable between a covered position where the cap at least partially surrounds the needle cover and the needle and an uncovered position where the needle cover or the needle are exposed. The cap can include one or more catch engageable with the one or more projections. The cap can include a pair of sidewall projections extending outward from a pair of sidewalls, respectively, of the cap.

### CLAIM OF PRIORITY

This application claims the benefit of priority under 35 U.S.C. §119(e) to U.S. Provisional Patent Application No. 63/769,493, filed on Mar. 10, 2025, which is incorporated by reference herein in its entirety.

### BACKGROUND

Medical instruments with a pointed needle are used extensively in various medical fields. For example, needles are used with syringes for delivery of medication or withdrawal of blood. These medical instruments can be used by medical professionals (e.g., doctors, nurses, or physicians) in clinics or hospitals, and can also be used by non-professionals at home, such as for insulin injections. Following use, needles should be carefully handled and disposed of to help avoid needle sticks, which can be an accidental penetration of skin or tissue of a person by the needle outside of the needle's intended use.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of a syringe and needle protection device.

FIG. 2 illustrates a perspective view of a syringe and needle protection device.

FIG. 3 illustrates a perspective view of a needle protection device.

FIG. 4 illustrates a perspective view of a needle protection device.

FIG. 5 illustrates a perspective view of a needle protection device.

FIG. 6 illustrates a perspective view of a needle protection device.

FIG. 7 illustrates a perspective view of a needle protection device.

FIG. 8 illustrates a perspective view of a needle protection device.

FIG. 9 illustrates a perspective view of a needle protection device.

FIG. 10 illustrates a perspective view of a needle protection device.

### DETAILED DESCRIPTION

Because syringes and other devices including needles or sharps are prevalent, it is known that it is desirable to avoid needle sticks outside of designed or designated use. Many devices include retractable needles; however, such devices can be relatively complex and expensive. Some devices include a cap securable to the syringe following use; however, such devices are typically not removable such that the items cannot be reused or used in a first instance if accidentally closed. Some devices that are reusable are not relatively secure.

The needle protection device discussed below helps to address these issues by providing a needle cap that is releasably securable to a needle to cover or surround the needle after use to help limit or prevent accidental needle sticks. The needle protection device can also be releasably securable to the syringe to allow for reuse (e.g., following cleaning) of one or more components of the assembly. The needle cap assembly can also have a design that can be relatively inexpensive to manufacture.

For example, a needle protection device can include a collar a needle cover and a cap. The collar can be securable to a hub or a barrel of a syringe, and the collar can include one or more projections. The needle cover can be releasably securable to the collar and can be configured to at least partially surround a needle of the syringe when the needle cover is secured to the collar. The cap can be pivotably connected to the collar and can be movable between a covered position where the cap at least partially surrounds the needle cover and the needle and an uncovered position where the needle cover or the needle are exposed. The cap can include catches engageable with projections of the collar to releasably secure the cap to the collar in the covered position.

The above discussion is intended to provide an overview of subject matter of the present patent application. It is not intended to provide an exclusive or exhaustive explanation of the invention. The description below is included to provide further information about the present patent application.

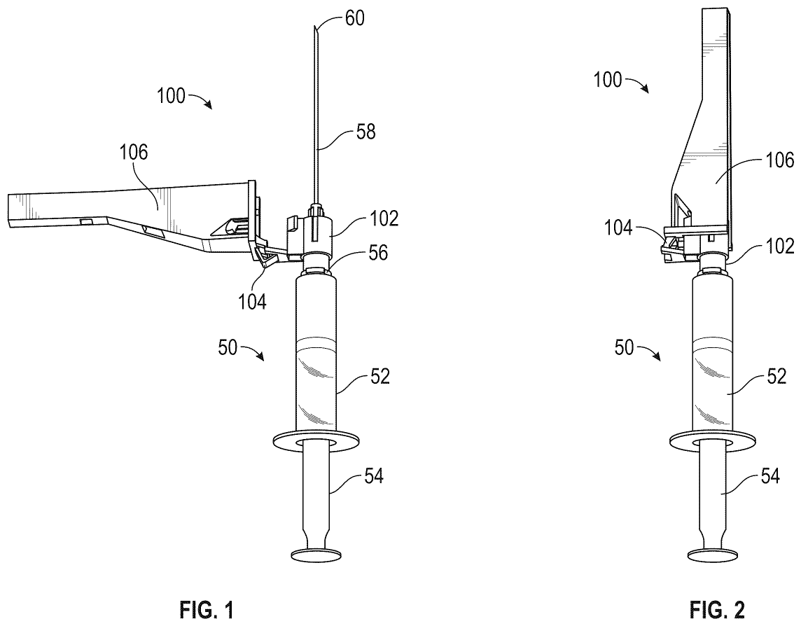

FIG. 1 illustrates a perspective view of a needle protection device 100. FIG. 2 illustrates a perspective view of the needle protection device 100. FIGS. 1 and 2 are discussed together below and also show a syringe 50, which can include a barrel 52, a plunger 54, a hub 56, and a needle 58 including a sharp 60 (or point or bevel and lumen).

The needle protection device 100 can include a collar 102, a needle cover (discussed below), a hinge 104, and a cap 106. The hinge 104 can be connected to the collar 102 and can be connected to the cap 106 to allow the cap 106 to move with respect to the collar 102 between a covered position and an uncovered position.

The collar 102 can be securable to the hub 52 or the barrel 56 of the syringe 50. The cap 106 can be pivotably connected to the collar 102 by the hinge 104 such that the cap 106 is movable between an uncovered position (as shown in FIG. 1) and a covered position (as shown in FIG. 2). In the covered position, the cap 106 can at least partially surround the needle 58 whereas the needle 58 can be exposed in the uncovered position. As discussed in further detail below, the cap 106 can be releasably secured to the collar 102 when the cap 106 is in the covered position.

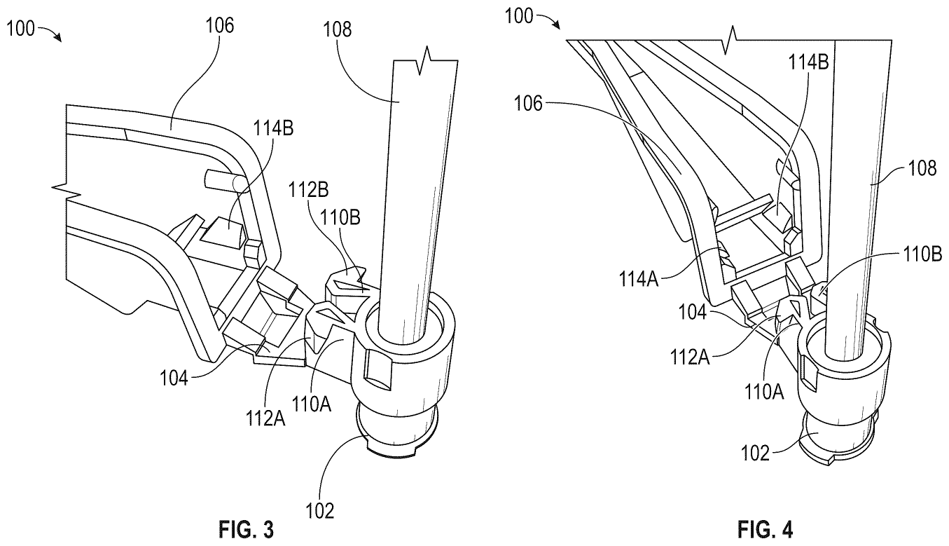

FIGS. 3 and 4 illustrate perspective views of the needle protection device 100 and are discussed together below. The needle protection device 100 of FIGS. 3 and 4 can be consistent with the needle protection device 100 of FIGS. 1 and 2 discussed above. FIGS. 3 and 4 show additional details of the needle protection device 100.

For example, FIGS. 3 and 4 show that the needle protection device 100 can include a needle cover 108. The needle cover 108 can be a cover or cap that can have a shape that is a hollow cylinder but can have other shapes in other examples, such as a hollow rectangular prism, or the like. The needle cover 108 can be releasably securable to the collar 102. The needle cover 108 can be configured to at least partially surround the needle 58 of the syringe 50 when the needle cover 108 is secured to the collar 102.

FIGS. 3 and 4 also show that the collar 102 can include one or more projections 110, such as a pair of projections 110a and 110b. The projections 110a and 110b can each include a barb 112a and 112b, respectively. The barbs 112 can extend laterally outward from a body of the projections 110 and the barbs 112a and 112b can be releasably securable to a respective catch 114 of the cap 106.

The catches 114a and 114b can be connected to or formed of the cap 106 and can be located on respective side walls 118 of the cap 106. The catches 114a and 114b can be engageable with the projections 110a and 110b, respectively, to cause the projections 110a and 110b to elastically deflect (e.g., laterally inward) to allow the barbs 112a and 112b, respectively, to move into the cap 106 past the catches 114a and 114b, respectively. The projections 110 can be biased to laterally outward positions such that once the barbs 112a and 112b move past the catches 114a and 114b, respectively, the projections 110 (and the barbs 112) can deflect (e.g., laterally outward) to engage an opposing side of the catches 114 to releasably secure the cap 106 to the collar 102 in the covered position.

When it is desired to open the cap 106 after securing the cap 106 to the projections 110 and collar 102, the projections 110 can be deflected inward (e.g., by a user) to allow the barbs 112 to move past the catches 114 so that the cap 106 can be moved back to the open position. In this way, the needle protection device 100 can be used to protect a user from needle sticks when the cap is in the closed position and the needle protection device 100 can allow the needle protection device 100 to be reused or the syringe 50 to be reused when appropriate.

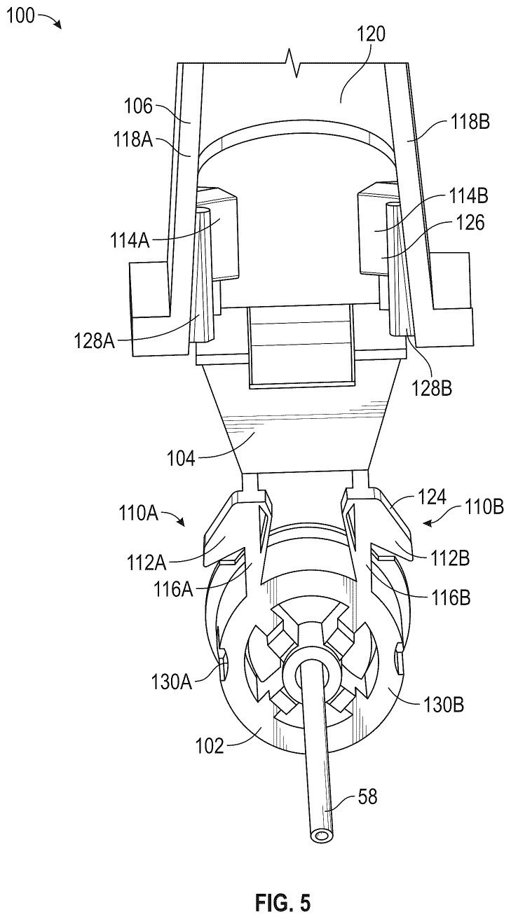

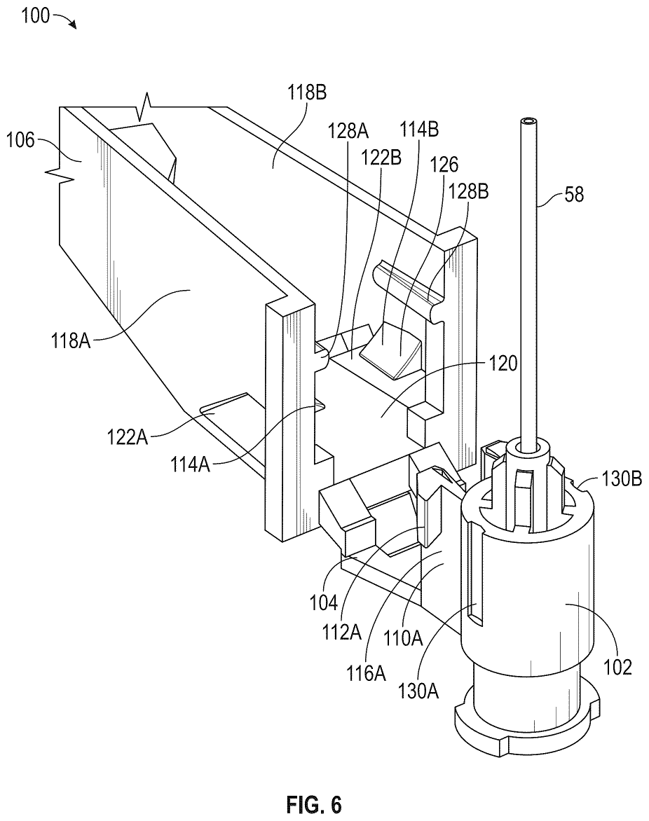

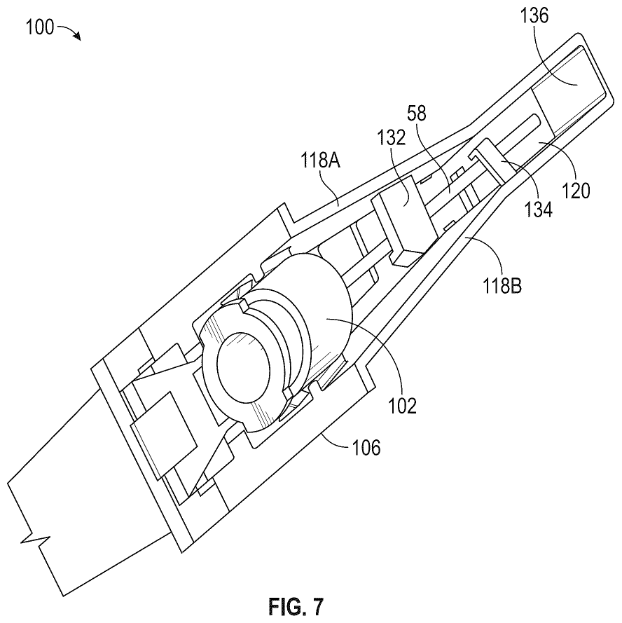

FIGS. 5 and 6 illustrate perspective views of the needle protection device 100 and are discussed together below. The needle protection device 100 of FIGS. 5 and 6 can be consistent with the needle protection device 100 of FIGS. 1-4 discussed above. FIGS. 5 and 6 show additional details of the needle protection device 100. For example, FIGS. 5 and 6 show additional details of the projections 110.

FIG. 5 most clearly shows that each of the projections 110a and 110b can include a body 116a and 116b, respectively, that can each extend laterally or radially outward from an outer surface of the collar 102. The barbs 112a and 112b can be connected to a distal or outer portion of the bodies 116a and 116b, respectively, and the barbs can extend perpendicularly or away from a direction of extension of the bodies 116a and 116b. The bodies 116 can allow the projections 110 to deflect, such as towards each other, to allow the projections 110 to move for fastening or securing to the catches 114. FIG. 6 also shows that the hinge 104 can be connected to the bodies 116 of the projections 110 to connect the cap 106 to the collar 102 via the hinge 104.

FIGS. 5 and 6 also show that the cap 106 can include a pair of sidewalls 118a and 118b and a rear wall 120. The pair of side walls 118 can be connected to opposite sides of the rear wall 120 to form an opening or cavity between the pair of side walls 118 and opposite the rear wall 120 such that the rear wall 120 and the pair of side walls 118 together are configured to at least partially surround the needle cover 108 and the needle 58 when the cap 106 is in the covered position, and such that the needle 58 is located at least partially within the cavity formed by the side walls 118 and the rear wall 120.

FIGS. 5 and 6 also show that the catches 114a and 114b can extend inward from the side walls 118a and 118b, respectively, into the cap 106. The catches 114 can be integral to, or formed of or formed from, the side walls 118, respectively. FIG. 6 more clearly shows that the catches 114 can be formed of the side walls 118. For example, FIG. 6 shows that the catch 114b can extend inward and can form an opening 122 in the sidewall 118b that can be configured to receive the projection 110b at least partially therein. The openings 122a and 122b can be adjacent respective ones of the catches 114a and 114b and can allow for access to the projections 110 when the cap 106 is in the locked positions such that the openings 122a and 122b can allow a user to apply forces to the projections 110 to force the projections 110 inward toward each other to disengage the projections from the catches 114 and to help allow the cap 106 to be moved to the open position.

FIG. 5 also shows that each of the barbs 112 can include an outer surface 124 or portion that can be profiled, such as chamfered, beveled, rounded, or curved. Such a profiled surface 124 can allow the projections 110 to deflect inward when the outer surface 124 engages a ramped surface 126 (shown more clearly in FIG. 6) of the catches 114 as the cap 106 is moved from the open position to a closed position.

FIGS. 5 and 6 also show that the cap 106 can include bosses or projections 128a and 128b that can extend inwards (e.g., laterally inward) from respective side walls 118a and 118b. For example, the bosses 128 can extend towards each other. FIGS. 5 and 6 also show that the collar 102 can include recesses 130a and 130b that can extend radially into opposing sides of the collar 102 from a radially outer surface of the collar 102. The recesses 130a and 130b can be configured to receive at least a portion of the bosses 128a and 128b when the cap 106 is in the covered position to secure the cap 106 in the covered position. The bosses 128 and the recesses 130 can interface in a friction fit such that the bosses 128 can be releasably securable to the recesses 130 and the collar 102. In this way, the cap 106 can be secured to the collar 102 using multiple features that are releasable or reversible.

The bosses 128 can be rounded or can have an outer surface or profile that is rounded. Such a rounded surface of the bosses 128 can reduce friction between the 128 and the 102, which can help the bosses 128 move along an outer surface of the collar 102 prior to or after insertion into or from the recesses 130.

FIG. 7 illustrates a perspective view of a needle protection device 100. The needle protection device 100 of FIG. 7 can be consistent with the needle protection device 100 of FIGS. 1-6 discussed above. FIG. 7 shows additional details of the needle protection device 100. For example, FIG. 7 shows that the needle protection device 100 can include one or more needle catches configured to engage the needle or the needle cover to help retain the needle when the cap is in the closed position.

More specifically, the cap 106 can include needle catches or retainers 132 and 134. The retainer 132 can be connected to and can extend inward from the sidewall 118a, such as toward the sidewall 118b, but can terminate before reaching the sidewall 118b. Similarly, the retainer 134 can be connected to and can extend inward from the sidewall 118b, such as toward the sidewall 118a, but can terminate before reaching the sidewall 118a. By being cantilevered and having a relatively small thickness, each of the needle catches 132 and 134 can be configured to deflect. For example, when the cap 106 moves from the open position to the closed position (shown in FIG. 7), the retainers 132 and 134 can engage the needle 58 or the needle cover 108 and can deflect toward the rear wall 120 to allow the needle 58 or the needle cover 108 to pass the retainers 132 and 134.

When the cap 106 is secured to the collar 102, the needle 58 and the needle cover 108 can be positioned between the retainers 132 and 134 and the rear wall 120 such that the needle 58 and the needle cover 108 are limited from moving out of the cap 106. Because the retainers 132 and 134 are configured to deflect, when the cap 106 is unsecured from the collar 102 (such as by manipulating the projections 110 to disengage the catches 114), the retainers 132 and 134 can deflect away from the rear wall 120 upon application of sufficient force to move the needle 58 and the needle cover 108 out of the cap 106. In this way, the needle 58 and the needle cover 108 can be limited from moving out of the cap 106 until the cap 106 is released from the collar 102 and therefore protected by the cap 106, such as from unintentional needle sticks between or after use of the syringe 50.

FIG. 7 also shows that the 106 can include a top wall 136 that can be connected to the side walls 118 and the rear wall 120 to form the cavity or opening of the cap 106 that receives the needle 58 at least partially therein when the cap 106 is in the closed position. The top wall 136 can especially help to limit or prevent needle sticks with the cap 106 is in the closed position.

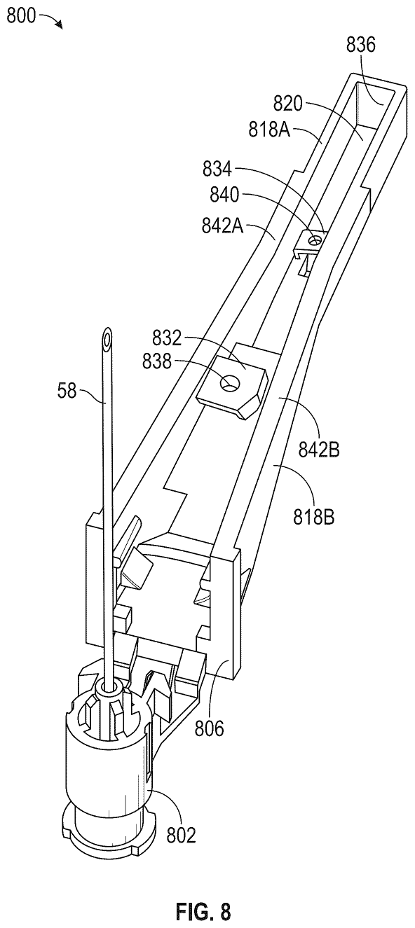

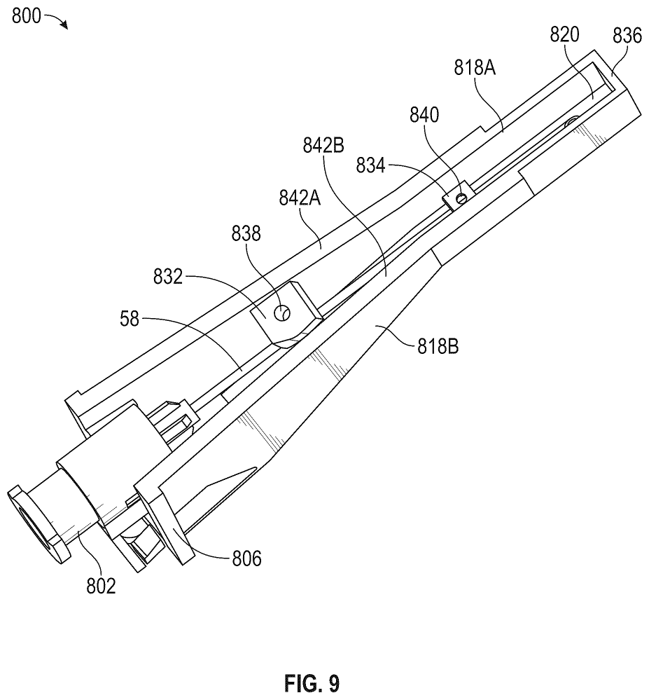

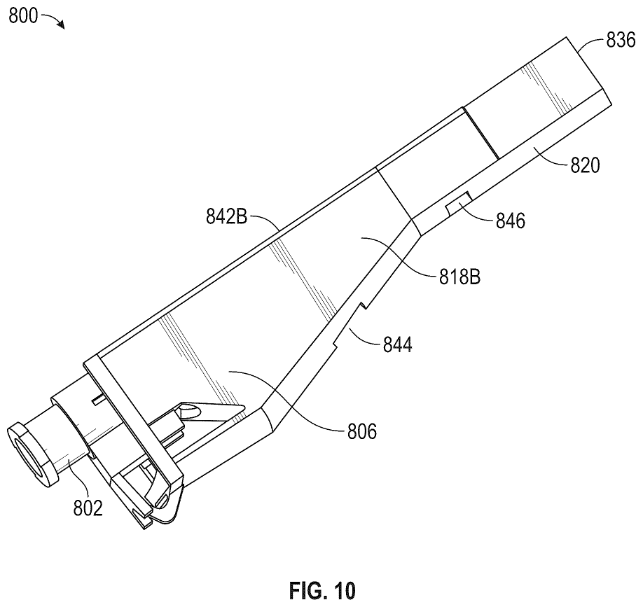

FIG. 8 illustrates a perspective view of a needle protection device 800 in an open position. FIG. 9 illustrates a perspective view of the needle protection device 800 in a covered or closed position. FIG. 10 illustrates a perspective view of the needle protection device 800 in a covered or closed position. FIGS. 8-10 are discussed together below. The needle protection device 800 of FIGS. 8-10 can be similar to the needle protection device 100 discussed above.

FIGS. 8 and 9 show that the needle protection device 800 can include one or more needle catches configured to engage the needle or the needle cover to help retain the needle when the cap is in the closed position. More specifically, a cap 806 can include needle catches or retainers 832 and 834. The retainer 832 can be connected to and can extend inward from a sidewall 818a, such as toward a sidewall 818b, but can terminate before reaching the sidewall 818b. Similarly, the retainer 834 can be connected to and can extend inward from the sidewall 818b, such as toward the sidewall 818a, but can terminate before reaching the sidewall 818a. By being cantilevered and having a relatively small thickness, each of the needle catches 832 and 834 can be configured to deflect. For example, when the cap 806 moves from the open position (shown in FIG. 8) to the closed position, the retainers 832 and 834 can engage the needle 58 or the needle cover 808 and can deflect toward the rear wall 820 to allow the needle 58 or the needle cover (e.g., the needle cover 108) to pass the retainers 832 and 834.

When the cap 806 is secured to the collar 802, the needle 58 and the needle cover 108 can be positioned between the retainers 832 and 834 and the rear wall 820 such that the needle 58 and the needle cover 08 are limited from moving out of the cap 106. Because the retainers 832 and 834 are configured to deflect, when the cap 106 is unsecured from the collar 802 (such as by manipulating projections to disengage catches), the retainers 832 and 834 can deflect away from the rear wall 820 upon application of sufficient force to move the needle 58 and the needle cover out of the cap 806. In this way, the needle 58 and the needle cover can be limited from moving out of the cap 806 until the cap 806 is released from the collar 802 and therefore protected by the cap 806, such as from unintentional needle sticks between or after use of the syringe 50.

FIGS. 8 and 9 also show that the 106 can include a top wall 836 that can be connected to the side walls 818 and the rear wall 820 to form the cavity or opening of the cap 806 that receives the needle 58 at least partially therein when the cap 806 is in the closed position. The top wall 836 can especially help to limit or prevent needle sticks with the cap 806 is in the closed position.

FIGS. 8 and 9 also show that the retainer 832 can include a bore or opening 838 and the retainer 834 can include a bore or opening 840. The bores can reduce an amount of material required during manufacturing and can help to increase visibility of the needle 58 when the needle 58 and the cap 806 are in the closed configuration (as shown in FIG. 9). Visibility of the needle 58 can allow for inspection of the needle 58 to ensure there are no breaks, which can help reduce need stick incidents.

FIGS. 8 and 9 also show that the cap 806 can include projections 842a and 842b extending from the sidewalls 818a and 818b, respectively. For example, as shown more clearly in FIG. 10, the projection 842b can extend outward from the sidewall 818b. The projections 842a and 842b can thereby be user-operated or user-graspable to more easily grasp an outer portion of the cap 806 and move the cap 806 to the closed or covered position. The projections 842a and 842b can also increase a rigidity of the cap 806.

FIG. 10 also shows, more clearly, that the cap 806 can include openings 844 and 846. The opening 844 can extend at least partially through the rear wall 820 at or near the retainer 832. Similarly, the opening 846 can extend at least partially through the rear wall 820 at or near the retainer 834. The openings 844 and 846 can thereby allow a user to inspect the needle 58 within the cap 806 from another perspective and can allow a user to confirm the retainers 832 and 834 are in place relative to the needle 58 when the cap 806 is moved to the closed position. FIG. 10 also shows that a rear portion or rear surface of the rear wall 820 can be curved or arced (in the shape of an arc) between the sidewalls 818, which can improve ergonomics and grasp-ability of the cap 806.

### NOTES AND EXAMPLES

The following, non-limiting examples, detail certain aspects of the present subject matter to solve the challenges and provide the benefits discussed herein, among others.

Example 1 is a needle protection device comprising: a collar securable to a hub or a barrel of a syringe, the collar including one or more projections; a needle cover releasably securable to the collar and configured to at least partially surround a needle of the syringe when the needle cover is secured to the collar; and a cap pivotably connected to the collar and movable between a covered position where the cap at least partially surrounds the needle cover and the needle and an uncovered position where the needle cover or the needle are exposed, the cap including one or more catch engageable with the one or more projections of the collar to connect the one or more projections to the one or more catch to secure the cap to the collar in the covered position, and the cap including a pair of sidewall projections extending outward from a pair of sidewalls, respectively, of the cap.

In Example 2, the subject matter of Example 1 optionally includes wherein the pair of sidewall projections are user-graspable to move the cap from the uncovered position to the covered position.

In Example 3, the subject matter of Example 2 optionally includes wherein the cap comprises: a rear wall connected to the pair of side walls, the side walls located on opposite sides of the rear wall to form an opening between the pair of side walls and opposite the rear wall, the rear wall and the pair of side walls together configured to at least partially surround the needle cover and the needle when the cap is in the covered position.

In Example 4, the subject matter of Example 3 optionally includes wherein each of the one or more catch includes a bore extending therethrough.

In Example 5, the subject matter of Example 4 optionally includes wherein the one or more catch are configured to deflect in response to engagement between the one or more catch and the needle or the needle cover such that the needle or the needle cover is located between the rear wall and the one or more catch when the cap is in the covered position.

In Example 6, the subject matter of Example 5 optionally includes wherein each of the one or more catch are integral to the cap.

In Example 7, the subject matter of any one or more of Examples 4-6 optionally include wherein a rear surface of the rear wall is curved between the sidewalls.

In Example 8, the subject matter of any one or more of Examples 3-7 optionally include wherein each of the one or more catch includes a bore extending therethrough.

In Example 9, the subject matter of Example 8 optionally includes wherein the one or more catch are configured to deflect in response to engagement between the one or more catch and the needle or the needle cover such that the needle or the needle cover is located between the rear wall and the one or more catch when the cap is in the covered position.

In Example 10, the subject matter of any one or more of Examples 1-9 optionally include a hinge connected to the collar and connected to the cap to allow the cap to move with respect to the collar between the covered position and the uncovered position.

Example 11 is a needle protection device comprising: a collar securable to a hub or a barrel of a syringe; a pair of projections extending outward from the collar; a needle cover releasably securable to the collar to at least partially surround a needle of the syringe; and a cap connected to the collar by a hinge, the cap movable between a covered position where the cap at least partially surrounds the needle cover or the needle and an uncovered position where the needle cover or the needle are exposed, the cap including a pair of catches engageable with the pair of projections to secure the cap to the collar in the covered position, and the cap including a pair of sidewall projections extending outward from a pair of sidewalls, respectively, of the cap.

In Example 12, the subject matter of Example 11 optionally includes wherein the pair of sidewall projections are user-graspable to move the cap from the uncovered position to the covered position.

In Example 13, the subject matter of Example 12 optionally includes wherein the cap comprises: a rear wall connected to the pair of side walls, the side walls located on opposite sides of the rear wall to form an opening between the pair of side walls and opposite the rear wall, the rear wall and the pair of side walls together configured to at least partially surround the needle cover and the needle when the cap is in the covered position.

In Example 14, the subject matter of Example 13 optionally includes wherein each of the pair of catches includes a bore extending therethrough.

In Example 15, the subject matter of Example 14 optionally includes wherein the pair of catches are configured to deflect in response to engagement between the pair of catches and the needle or the needle cover such that the needle or the needle cover is located between the rear wall and the pair of catches when the cap is in the covered position.

In Example 16, the subject matter of Example 15 optionally includes wherein each of the pair of catches are integral to the cap.

In Example 17, the subject matter of any one or more of Examples 14-16 optionally include wherein a rear surface of the rear wall is curved between the sidewalls.

In Example 18, the subject matter of any one or more of Examples 13-17 optionally include wherein each of the pair of catches includes a bore extending therethrough.

In Example 19, the subject matter of Example 18 optionally includes wherein the pair of catches are configured to deflect in response to engagement between the pair of catches and the needle or the needle cover such that the needle or the needle cover is located between the rear wall and the pair of catches when the cap is in the covered position.

In Example 20, the subject matter of any one or more of Examples 11-19 optionally include a hinge connected to the collar and connected to the cap to allow the cap to move with respect to the collar between the covered position and the uncovered position.

In Example 21, the apparatuses or method of any one or any combination of Examples 1-20 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A needle protection device comprising:
a collar securable to a hub or a barrel of a syringe, the collar including one or more projections;
a needle cover releasably securable to the collar and configured to at least partially surround a needle of the syringe when the needle cover is secured to the collar; and
a cap pivotably connected to the collar and movable between a covered position where the cap at least partially surrounds the needle cover and the needle and an uncovered position where the needle cover or the needle are exposed, the cap including one or more catch engageable with the one or more projections of the collar to connect the one or more projections to the one or more catch to secure the cap to the collar in the covered position, and the cap including a pair of sidewall projections extending outward from a pair of sidewalls, respectively, of the cap.

2. The needle protection device of claim 1, wherein the pair of sidewall projections are user-graspable to move the cap from the uncovered position to the covered position.

3. The needle protection device of claim 2, wherein the cap comprises:
a rear wall connected to the pair of side walls, the side walls located on opposite sides of the rear wall to form an opening between the pair of side walls and opposite the rear wall, the rear wall and the pair of side walls together configured to at least partially surround the needle cover and the needle when the cap is in the covered position.

4. The needle protection device of claim 3, wherein each of the one or more catch includes a bore extending therethrough.

5. The needle protection device of claim 4, wherein the one or more catch are configured to deflect in response to engagement between the one or more catch and the needle or the needle cover such that the needle or the needle cover is located between the rear wall and the one or more catch when the cap is in the covered position.

6. The needle protection device of claim 5, wherein each of the one or more catch are integral to the cap.

7. The needle protection device of claim 4, wherein a rear surface of the rear wall is curved between the sidewalls.

8. The needle protection device of claim 3, wherein each of the one or more catch includes a bore extending therethrough.

9. The needle protection device of claim 8, wherein the one or more catch are configured to deflect in response to engagement between the one or more catch and the needle or the needle cover such that the needle or the needle cover is located between the rear wall and the one or more catch when the cap is in the covered position.

10. The needle protection device of claim 1, comprising:
a hinge connected to the collar and connected to the cap to allow the cap to move with respect to the collar between the covered position and the uncovered position.

11. A needle protection device comprising:
a collar securable to a hub or a barrel of a syringe;
a pair of projections extending outward from the collar;
a needle cover releasably securable to the collar to at least partially surround a needle of the syringe; and
a cap connected to the collar by a hinge, the cap movable between a covered position where the cap at least partially surrounds the needle cover or the needle and an uncovered position where the needle cover or the needle are exposed, the cap including a pair of catches engageable with the pair of projections to secure the cap to the collar in the covered position, and the cap including a pair of sidewall projections extending outward from a pair of sidewalls, respectively, of the cap.

12. The needle protection device of claim 11, wherein the pair of sidewall projections are user-graspable to move the cap from the uncovered position to the covered position.

13. The needle protection device of claim 12, wherein the cap comprises:
a rear wall connected to the pair of side walls, the side walls located on opposite sides of the rear wall to form an opening between the pair of side walls and opposite the rear wall, the rear wall and the pair of side walls together configured to at least partially surround the needle cover and the needle when the cap is in the covered position.

14. The needle protection device of claim 13, wherein each of the pair of catches includes a bore extending therethrough.

15. The needle protection device of claim 14, wherein the pair of catches are configured to deflect in response to engagement between the pair of catches and the needle or the needle cover such that the needle or the needle cover is located between the rear wall and the pair of catches when the cap is in the covered position.

16. The needle protection device of claim 15, wherein each of the pair of catches are integral to the cap.

17. The needle protection device of claim 14, wherein a rear surface of the rear wall is curved between the sidewalls.

18. The needle protection device of claim 13, wherein each of the pair of catches includes a bore extending therethrough.

19. The needle protection device of claim 18, wherein the pair of catches are configured to deflect in response to engagement between the pair of catches and the needle or the needle cover such that the needle or the needle cover is located between the rear wall and the pair of catches when the cap is in the covered position.

20. The needle protection device of claim 11, comprising:
a hinge connected to the collar and connected to the cap to allow the cap to move with respect to the collar between the covered position and the uncovered position.
